(19) United States
(12) Patent Application Publication
(10) Pub. No.: US 2007/0281991 A1
(43) Pub. Date: Dec. 6, 2007

(54) Preparation Of Phenol-Amide Compounds With Anti-Oxidizing Properties

(76) Inventors: Guy Adrian, Brignais (FR); Patrick Bigot, Lyon (FR)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 11/575,729

(22) PCT Filed: Sep. 22, 2005
(86) PCT No.: PCT/FR05/02351

§ 371 Date: Jul. 3, 2007

(30) Foreign Application Priority Data
Sep. 24, 2004 (FR) 0410098

(51) Int. Cl. A61K 31/352; A61K 31/165; A61P 17/00; A61P 35/00; C07C 233/00; C07C 235/48; C07D 311/00; C07D 311/02

(52) U.S. Cl. 514/456; 514/617; 514/622; 549/405; 564/177

(57) ABSTRACT

The invention relates to the preparation of compounds comprising at least one phenol function and one amide function derived from amino-2-alkanol-1 having formula (I) and (II), which have antioxidant and an tiradical properties and which are soluble in lipid media. The inventive compounds can be used as cosmetic or pharmaceutical preparations for the prevention of biological degradation caused by free radicals.

The present invention concerns the preparation of phenol-amide compounds with anti-oxidizing properties derived from aromatic or heteroaromatic acids including at least one hydroxy group and 2-amino-1-alcanols corresponding to formulas (I) and (II): 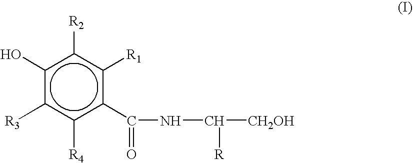
where R₁, R₂, R₃, R₄=—OH, HS or alkyl (CₙH₂ₙ₊₁), n being included between 1 and 4, R=alkyl (—CₙH₂ₙ₊₁) or hydroxy-alkyl (—CₙH₂ₙ₊₁O), n being included between 2 and 30. 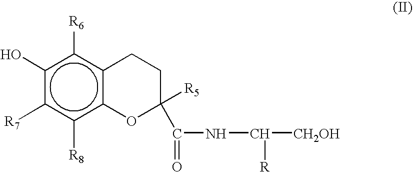
where R₅=alkyl (—CₙH₂ₙ₊₁), n being included between 1 and 4, where R₆, R₇, R₈=—OH, H or alkyl (—CₙH₂ₙ₊₁), n being included between 1 and 4, R=alkyl or hydroxy alkyl with the same structure as in formula (I).

Compounds (I) and (II), amides derived from 2-amino-1-alcanols, present some antiradical anti-oxidizing properties and can be used in the prevention of degenerative illnesses and human ageing: appearance of the skin (wrinkles), cardio-vascular illnesses, neuro-degenerative illnesses and cancers, while efficiently capturing the free radicals that damage the cells and tissues.

Indeed, the antioxidants, for example α-tocopherol (vitamin E), play a pivotal role in inhibiting the deterioration of DNA, proteins and lipids while reacting with the free radicals before they attack the substrata and while interrupting the propagation of radical chains of oxidization (B. Halliwell and J. Gutteridge—Free Radicals in Biology and Medicine—1998).

Among natural anti-oxidizing compounds, many derivatives of polyphenols are used, particularly derivatives of gallic acid (III)—3,4,5-trihydroxy benzoic acid (gallic acid) and its methylic ester (IIIa).

Derivatives of 6-dihydroxy-dihydrochroman (IV) are also used, where R₅, R₆, R₇, R₈ are alkyl chains and particularly CH₃, and X is an oxygenated group, for example acyl.

TROLOX (IVa) is a very well known example of this family of compounds. (J. W. Scott, Journal of the American Oil Chemist's Society 1974, 51, p. 200).

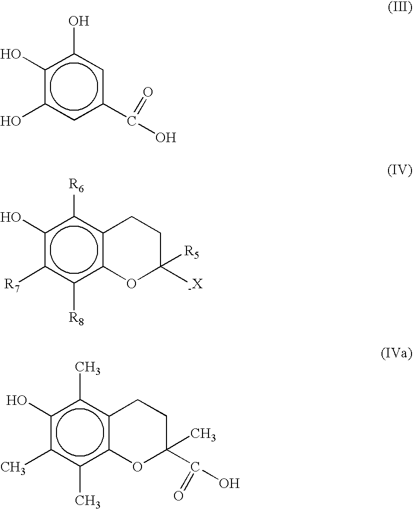

The aforementioned antioxidants, notably (III), (IIIa) and (Iva), are active compounds in the medium of polar solvents such as water or alcohol but less efficient in lipidic media (Halliwell and Gutteridge).

The goal of this invention is to obtain stable and non-toxic mixable anti-oxidizing structures in lipidic media, in particular in the optics of a protective effect at the level of the dermis by penetrating the skin to the level of the stratum corneum.

The 2-amino-1-alcanols (V), where R represents a linear or branched alkyl chain or hydroxyalkyl including 2 to 30 atoms of carbon, are prepared by methods known to professionals: for example by the reduction of α-amino racemic acids obtained by substitution with the ammonia of the commercially corresponding α-halogen acids.

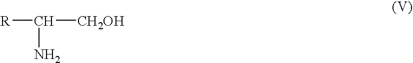

The acylation reaction of the amino function of 2-amino-1-alcanol (V) permits favorable attainment of type (I) and (II) compounds, presenting an efficient penetration at the level of the cutaneous tissues and a weak toxicity.

For the aforementioned uses, 2-amino-1-dodecanol is preferred, where R=decyl (n-C₁₀H₂₁) leading to the amines (Ia) and (IIa).

The formation of amides from the 2-amino-1-alcanols (V) is performed by activation of the carboxyl functions of acids (III) or (IV) to obtain an amide function, for example by forming additive compounds on dicyclohexylcarbodiimide in the presence of hydroxy benzotriazole according to Chemische Berichte 1970, Vol. 103, p. 788-798.

The presence of free hydroxy groups in acids (III) or (IV) leads to the impure products (I) and (II) containing O-acylated derivatives of phenol, which require purification by chromatography under pressure.

One would prefer protection of the phenol functions by acetic anhydride to obtain the O-acylated acids (VI) and (VII), then passage to the chlorides of corresponding acids of an agent of halogenation and particularly thionyl chloride, then selective condensation of the chlorides of O-acylated aromatic acids on the amino function of the aminoalcanols (V).

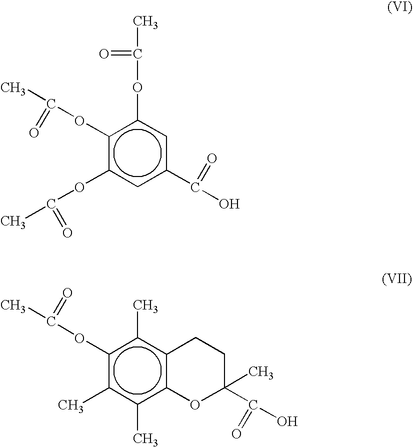

Compounds (I) and (II) were obtained after selective hydrolysis of the acetoxy groups by a basic agent and, in particular, cod sodium hydroxide.

Compounds (I) and (II) were isolated according to the appropriate techniques and purified by crystallization or column chromatography by silica.

The anti-oxidizing potential in solution of the amide-phenol compounds (I) and (II) was evaluated by the degree of their reaction with radical free diphenyl-picrylhydrazide (VIII) following C. T. Ho, Journal of Agricultural and Food Chemistry, 1999, p. 3975, while using TROLOX (IVa) and gallate of methyl (IIIa) as evidence that compounds (I) and (II) possess an activity similar to that of the products in question.

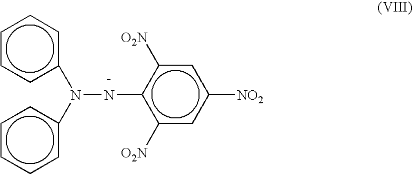

The applicant finalized a process of preparation of phenol-amides compounds with anti-oxidizing properties; these compounds can be shaped into tablets, capsules, or in a mixture in cosmetic preparations according to common practice.

Of course, various modifications can be introduced by experts to the devices or processes that have just been described solely as a non-restrictive example, without going beyond the bounds of the invention.

The invention is illustrated by the following examples 1 through 3:

### EXAMPLE 1

Obtaining the amide by formula (I) R=nC₁₀H₂₁ from gallic acid=3,4,5-hydroxybenzoic acid (III).

A solution of 2.55 g (15 mmoles) of gallic acid, of 4 g (30 mmoles) of hydroxybenzotriazole and of 3 g (10 mmoles) of 2-amino-1-dodecanol in 30 ml of tetrahydrofurane is cooled to 0° C., then a solution of 3.1 g (15 mmoles) of dicyclohexylcarbodiimide in 5 ml of THF is added in once.

After one hour of agitation at 0° C., then 2 hours at 20° C., the reactional medium is filtered. 3 g of dicyclohexylurea is collected. The filtrant is concentrated under vacuum, redissolved in 100 ml of methyl tertiobutyl ether and cleaned 4 times in 30 ml of hydrochloric acid 1N, then in 30 ml of an aqueous solution of 10% of baking soda, then twice in 30 ml of water and concentrated under vacuum to provide 3.8 g of a solid paste containing by RMN¹H about 70% of the expected amide that could not be purified by crystallization in the current solvents.

### EXAMPLE 2

Obtaining the amide by formula (Ia), R=nC₁₀H₂₁ from 3,4,5-triacetoxy benzoic acid.

One prepares a solution of 5.9 g (20 mmoles) of 3,4,5-triacetoxy benzoic acid F=170° C. (Berichte der Deutschen Chemiscbe Gesellschaft 1919, Vol. 52, p. 829-833) in 20 ml of 1,2-dichloroethane at 20° C.

1.73 g (24 mmoles) of thionyl chloride and 0.1 ml of triethylamine are dripped in. This medium is heated to reflux for 2 hours, then concentrated under reduced pressure.

The raw chloride acid is redissolved in 20 ml of 1,2-dichloroethane and 3.8 g (19 mmoles) of 2-amino-1-dodecanol is added at 20° C. followed by 27 ml (19 mmoles) of triethylamine.

The mixture is then heated to 50° C. for 18 hours under agitation and cooled down to 20° C. and washed with 100 ml of water.

The organic phase of 40 ml of ethanol, 30 ml of water and 5.6 g of an aqueous solution of sodium carbonate at 50% is added.

The agitated mixture is brought to 60° C. for 1 hour and then cooled down to 20° C., concentrated under vacuum to eliminate the ethanol and recovered by 100 ml of methyl tertiobutyl ether.

The medium is acidified to pH=2 by an aqueous solution of N hydrochloric acid, washed in water and by an aqueous solution of baking soda.

After concentration of the organi phase, a solid paste is obtained which, by agitation with 100 ml of water, crystallizes to yield 5.7 g of a beige solid that, after recrystallization in 50 ml of acetic ether, provides 2.6 g of a solid white cream F=110° C.

Rdt=38.8% from the 2-amino-1-dodecanol.

The RMN¹H spectrum at 250 MHz's in the dimethyl sulfoxide shows 5 exchangeable protons, 2 aromatic protons at δ=6.8 ppm and a set of aliphatic protons between 0.8 and 1.8 ppm compatible with the structure (1).

The mass spectrum shows a peak (M+1)=354.

### EXAMPLE 3

Obtaining the amide (IIa), R=nC₁₀H₂₁, R₅=R₆=R₇=R₈=CH₃.

A solution of 2.5 g (10 mmoles) of TROLOX (6-hydroxy-2,5,7,8-tetramethyl chroman-2-carboxylic acid) in 10 ml of 1,2-diclioroethane is mixed with 2 ml of acetic anhydride and 10 μl of triethylamine.

The mixture is heated for 3 hours at 60° C. then concentrated under vacuum. The residue is dissolved in 10 ml of dichloroethane.

2 ml of thionyl chloride are added; it is brought to reflux for 2 hours, and it is then concentrated in dry form and redissolved in 10 ml of dichloroethane.

A solution of 2.04 g (10 mmoles) of 2-amino-1-dodecanol and 1.4 ml of triethylamine in 5 ml of dichloroethane is added and this medium is brought to 50° C. under agitation for 18 hours and then concentrated again.

10 ml of ethanol and 10 ml of sodium carbonate 2N were added to the above raw mixture.

This medium was brought to 60° C. for 1 hour, then cooled down to 20° C., diluted by 20 ml of methyl tertiobutyl ether, and cleaned 3 times in 10 ml of N hydrochloric acid, 10 ml of water and twice in 10 ml of baking soda solution at 10%.

The organic phase was separated, dried on magnesium sulfate and concentrated in a rotary evaporator to obtain 2.7 g of a white wax that was chromatographied on 60 g of Fluka silica (40-60 micrometers) by means of an eluant mixture of 3 parts of cyclohexane to 2 parts of ethyl acetate to obtain 1.35 g.

Rdt=31%.

The RMN¹H spectrum at 250 MHz's in dimethyl sulfoxide showed 3 exchangeable protons, 4 groups of CH₃ of 2.0-2.1-2.6 and 3.2 ppm and aliphatic protons between 0.8 and 1.6 ppm.

The mass spectrum showed a main peak (M+1)=434.

The inhibitory effect of the free radicals claimed for the compounds (Ia) and (IIa) has been evaluated by the measure of their absorbencies in UV spectrophotometry at 517 nanometers in the presence of an excess of radical diphenylpicrylhydrazide (VIII).

Ethanolic solutions containing 20 micromoles by liter of compound to test and 100 micromeres by liter of radical (VIII) agitated for 30 minutes at 20° C. and placed in a vat 1 cm of length.

| Product tested | Absorbance at 517 nm | Inhibition % |
|---|---|---|
| Control only | 1.15 | |
| diphenylpicrylhydrazide | | |
| Gallate of methyl (IIIa) | 0.33 | 71 |
| Gallic amide (Ia) | 0.35 | 70 |
| TROLOX (IVa) | 0.10 | 91 |
| TROLOX amide (IIa) | 0.40 | 65 |

The new compounds (Ia) and (IIa) present an inhibitory activity lower than TROLOX but comparable to that of methyl gallate.

1. A compound of formula (I): 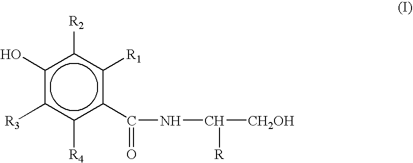
wherein:
R₁, R₂, R₃, and R₄ are —OH, —H or C₁₋₄alkyl; and
R is C₂₋₃₀alkyl.

2. (canceled)

3. (canceled)

4. (canceled)

5. (canceled)

6. (canceled)

7. The compound of claim 1, wherein the compound is derived from an aromatic acid including at least one hydroxy group and a 2-amino-1-alcanol.

8. A composition comprising a compound of formula (I): 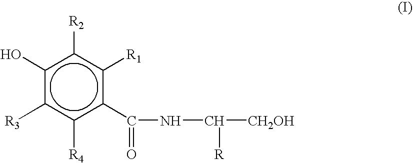
wherein R, R₁, R₂, R₃, and R₄ are as previously defined and the compound is prepared by a process comprising the steps of:
(a) cooling a solution of gallic acid, hydroxybenzotriazole and 2-amino-1-dodecanol to about 0° C.;
(b) adding to the solution of step (a) a solution of dicyclohexylcarbodiimide and tetrahydrofuran to form a reaction medium; and
(c) obtaining the compound of formula (I) from the reaction mixture.

9. A composition comprising a compound of formula (I): 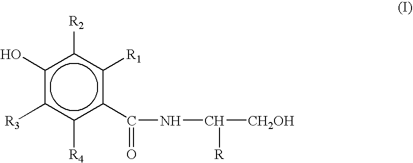
wherein R, R₁, R₂, R₃, and R₄ are as previously defined and the compound is prepared by a process comprising the steps of:
(a) preparing a solution of step 3,4,5-triacetoxybenzoic acid, dichoroeyhane, thionyl chloride and triethylamine;
(b) adding to the solution of step (a) additional dichloroethane, 2-amino-1-dodecanol and additional triethylamine to form a reaction mixture; and
(c) obtaining the compound of formula (I) from the reaction mixture.

10. The composition of claim 8, wherein the 2-amino-1-dodecanol of the process has an alkyl or hydroxyl-C₂-C₃₀alkyl.

11. The composition of claim 9, wherein the 2-amino-1-dodecanol of the process has an alkyl or hydroxyl-C₂-C₃₀alkyl.

12. The composition of claim 10, wherein the process further comprises the steps of:
(a) adding an aqueous solution of sodium carbonate to the reaction mixture;
(b) acidifying the reaction mixture;
(c) seperating from the acidified reaction mixture an aqueous phase and an organic phase;
(d) concentrating the organic phase; and
(e) crystallizing the compound from the concentrated organic phase.

13. The composition of claim 11, wherein the process further comprises the steps of:
(a) adding an aqueous solution of sodium carbonate to the reaction mixture;
(b) acidifying the reaction mixture;
(c) seperating from the acidified reaction mixture an aqueous phase and an organic phase;
(d) concentrating the organic phase; and
(e) crystallizing the compound from the concentrated organic phase.

14. Use of a compound of claim 1 for the manufacture of a cosmetic preperation or a pharmaceutical preperation for the prevention of biological deterioration due to free radicals.

15. A compound of formula (II): 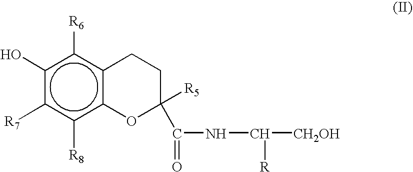
wherein:
R₅ is C₁-C₄alkyl;
R₆, R₇, R₈ are —OH, —H or C₁-C₄alkyl; and
R is R is C₂₋₃₀alkyl.

16. The compound of claim 15, wherein the compound is derived from an aromatic acid including at least one hydroxy group and a 2-amino-1-alcanol.

17. A composition comprising a compound of formula (II): 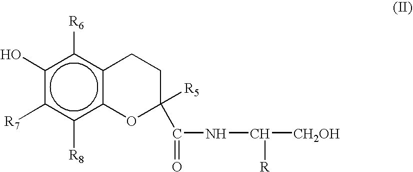
wherein the R, R₅, and R₆ are as previously defined and the compound is prepared by a process comprising the steps of:
(a) cooling a solution of a 6-hydroxy-dihydrochroman derivative, hydroxybenzotriazole and 2-amino-1-dodecanol to about 0° C.;
(b) adding to the solution of step (a) a solution of dicyclohexylcarodiimide and tetrahydrofuran to form a reaction mixture; and
(c) obtaining the compound of formula (II) from the reaction mixture.

18. A composition comprising a compound of formula (II): 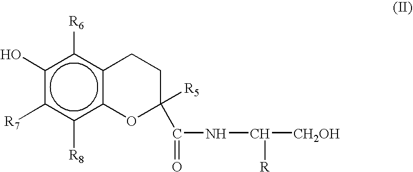
wherein R, R₁, R₂, R₃, and R₄ are as previously defined and the compound is prepared by a process comprising the steps of:
(d) preparing a solution of 3,4,5-triacetoxybenzoic acid, dichoroeyhane, thionyl chloride and triethylamine;
(e) adding to the solution of step (a) additional dichloroethane, 2-amino-1-dodecanol and additional triethylamine to form a reaction mixture; and
(f) obtaining the compound of formula (II) from the reaction mixture

19. The composition of claim 17, wherein the 2-amino-1-dodecanol of the process has an alkyl or hydroxyl-C₂-C₃₀alkyl.

20. The composition of claim 18, wherein the 2-amino-1-dodecanol of the process has an alkyl or hydroxyl-C₂-C₃₀alkyl.

21. The composition of claim 19, wherein the process further comprises the steps of:
(f) adding an aqueous solution of sodium carbonate to the reaction mixture;
(g) acidifying the reaction mixture;
(h) seperating from the acidified reaction mixture an aqueous phase and an organic phase;
(i) concentrating the organic phase; and
(j) crystallizing the compound from the concentrated organic phase.

22. The composition of claim 20, wherein the process further comprises the steps of:
(f) adding an aqueous solution of sodium carbonate to the reaction mixture;
(g) acidifying the reaction mixture;
(h) seperating from the acidified reaction mixture an aqueous phase and an organic phase;
(i) concentrating the organic phase; and
(a) crystallizing the compound from the concentrated organic phase.

23. Use of a compound of claim 15 for the manufacture of a cosmetic preperation or a pharmaceutical preperation for the prevention of biological deterioration due to free radicals.
